(19) United States
(12) Patent Application Publication
(10) Pub. No.: US 2023/0329297 A1
(43) Pub. Date: Oct. 19, 2023

(54) Method for Producing a Pumpable Paste from the Seeds of Nut or Stone Fruits

(71) Applicant: NETZSCH-Feinmahltechnik GmbH, Selb (DE)

(72) Inventors: Matheus Weidgenant, Selb (DE); Matthias Kapp, Weilersbach (DE)

(21) Appl. No.: 18/299,901

(22) Filed: Apr. 13, 2023

(30) Foreign Application Priority Data
Apr. 14, 2022 (DE) 10 2022 109 275.5

(51) Int. Cl. A23L 25/00; A23N 5/00; A23N 12/08; A23L 3/48

(52) U.S. Cl. CPC A23L 25/30; A23N 5/008; A23N 12/08; A23L 3/485

(57) ABSTRACT

A method for preparing a pumpable batch composition of stone fruit and/or nut fruit seeds, in particular nuts, almonds or nut and almond mixtures, with the following steps: Breaking open of the already pitted or stoned stone-fruit or nut-fruit seeds first takes place by pre-crushing. A hot air and/or vacuum exposure of the composition arising from the pre-crushing then takes place for the purpose of breaking open its cells. This is followed by a usually single-stage, optionally also multi-stage, further grinding of the composition arising from the pre-crushing and the hot air or vacuum exposure to form a pumpable batch composition.

### TECHNICAL FIELD

The invention relates to a method for preparing a pumpable batch composition of stone fruit and/or nut fruit seeds, in particular nuts, almonds or nut and almond mixtures.

### BACKGROUND

Almond-containing products including, in particular, almond-containing confectionery products, such as tarts, cakes, chocolates, Petit Fours, ice cream, marzipan and almond-containing biscuits have traditionally enjoyed great popularity. Whole or chopped almonds are usually used only for decorative purposes. Almond paste is required much more frequently for such products.

Moreover, almond-containing products have long been used in the cosmetics sector, not only in the form of skin-care almond oil or skin-purifying almond meal, but time and again also in the form of finely ground skin-care almond pastes.

The demand for almond-containing products has escalated in the past few years with the strong boom in the vegan movement and at the same time the increased diagnosis of cases of lactose intolerances and milk protein allergies. This is due not least to the fact that, with the aid of very finely ground almond pastes and the protein and fat contained therein, an emulsion can be produced with the addition of water which, optically, sensorily and in terms of taste, is reminiscent of milk and therefore increasingly sells as a corresponding substitute.

Since almond milk is “parve” (neutral, i.e. neither milk-containing nor meat-containing), it also has some relevance in the context of kashrut, i.e. the Jewish dietary law.

What is referred to in everyday language as “almond” is the seed of the almond fruit forming a so-called stone fruit. In the latter, the seed is surrounded by a woody core, the so-called almond core.

In the fields of use described up to this point, however, not only stone fruits, such as almond fruit and its almonds, are used, but to a similar extent also nut fruits, i.e. fruits in which the seed is surrounded by a hard woody outer shell.

In all of this, products from the seeds of stone and nut fruits are not least in demand from industrial food production as finely ground, pumpable intermediate products, which are referred to as batch compositions.

A pumpable intermediate product of the stated type can easily be produced in all those cases in which in which it does not cause disruption if the finished intermediate product contains water or oil of a different origin added to it to improve its flowability and possibly also emulsifiers not peculiar to stone fruits or not peculiar to nut fruits.

For reasons of flavour and/or for reasons of better acceptance by consumers, however, the respective intermediate product should increasingly manage without additives which do not originate from the indehiscent fruits or their seeds. The addition of water for improved flowability is also not without problems, since every addition of water is accompanied by the risk of contamination with germs. Even if no contamination with germs takes place via the water, a higher water content promotes the growth of germs possibly present in the intermediate product for other reasons.

In the past, therefore, methods have been established for the processing of almonds or nuts, which employ roasting of the almonds or nuts, which is followed by the grinding of the almonds or nuts. The natural water content of the almonds or nuts is reduced by the roasting. The roasting heat causes the cell walls to break open and thus releases the fat stored in the interior of the respective cell. A pumpable composition that is easier to further process can thus be obtained directly following the roasting.

However, the roasting is almost always accompanied by a more or less marked change in colour to brownish. Such a change in colour is disruptive not least in the production of milk substitute products, which in many cases should exhibit the white familiar with milk products as far as possible.

Moreover, the roasting of the almonds or nuts is almost inevitably also accompanied by a more or less marked Maillard reaction, i.e. a complex totality of reactions taking place beside one another and/or after one another, which leads to a plurality of reaction products, which influence the flavour or which are rejected by the consumer, since it is known that mutagenic or carcinogenic reaction products can be found in these reaction products—at all events in the case of processes conducted only sub-optimally.

### PROBLEM OF THE INVENTION

By contrast, it is the problem of the invention to specify a method for the production of a pumpable batch composition of ground stone fruit seeds or nut fruit seeds, which manages without the addition of non-stone-fruit or non-nut-fruit seed ingredients and has no influence or only a slight influence on the flavour of the batch composition.

### SUMMARY

The solution according to the invention is a method for preparing a pumpable batch composition of stone fruit and/or nut fruit seeds, in particular nuts, almonds or nut and almond mixtures, with the following steps:

Breaking open of the already pitted or stoned stone-fruit or nut-fruit seeds first takes place by pre-crushing.

A hot air and/or vacuum exposure of the composition arising from the pre-crushing then takes place for the purpose of breaking open its cells.

This is followed by a usually single-stage, optionally also multi-stage, further grinding of the composition arising from the pre-grinding and the hot air or vacuum exposure to form a pumpable batch composition.

As a result of the pre-crushing, the free surface of the stone fruit or nut fruit seeds (so-called pre-crushing material) subsequently exposed to the vacuum and/or the hot air is increased many times over. In this way, it is possible by applying a vacuum and/or by applying hot air to cause the cell walls of the cells of the pre-grinding material, which have largely remained intact, to split open. The fats or oil constituents stored in the intact cells are thus unlocked. The fats or oil constituents are mobilised in such a way that they significantly increase the viscosity of the pre-crushing material and thus make the pre-crushing material pumpable.

A “batch composition” is understood to mean a composition of the natural products discussed here, which are brought to the consumer as a commercial product and are suitably packaged, possibly stored and as required—preferably with the aid of a metering pump—can be mixed with further ingredients and together with the latter further processed to form finished products, such as pastries, ice cream or care products. The primary purpose of the batch composition may be to endow the finished products with the characteristic flavour of the raw products from which the batch composition is produced.

### PREFERRED FURTHER POSSIBLE DEVELOPMENTS

The hot air or vacuum exposure is preferably controlled via the exposure time, in such a way that the residual moisture of the pre-crushing material is reduced to less than 2.1% and preferably to less than 1.85%. The pre-crushing initiating or preceding the method also contributes significantly to achieving this aim. This is because, as a result of the splitting open of the cell walls thus promoted under the influence of the vacuum or the hot air, apart from the fats and oil constituents the water content stored in the cells is also released and can easily be removed. The reduction in the water content contributes significantly to improved stability of the finished batch composition, since compositions with as low a water content as possible are a poor breeding ground for spores and germs. Since, according to the invention, the desired pumpability takes place via the mobilisation of the fats and oil constituents hitherto enclosed in the cells, the further withdrawal of water does not hinder the achievement of the desired pumpability.

The air temperature of the hot air used for the drying preferably lies in the region of over 60° C. and ideally in the region of over 75° C. Only with these temperatures is splitting open of the cell walls guaranteeing sufficiently rapid, practically reasonable process durations achieved.

It is particularly preferable that the air temperature of the hot air used for the drying lies in the region of less than 100° C. and ideally in the region of less than 90° C. The process temperature thus lies below or markedly below the threshold of approximately 140° C., from which the Maillard reactions begin to take place, i.e. “roasting processes” take place. Thus, neither discolorations nor the roasting aromas typical of roasting and certainly desired in other connections than the one here arise in the course of the hot air application.

Attention is paid in particular to the fact that the hot air used for the drying flows through the drying area in such a way that it is conducted away from there still while the drying process continues to progress, as soon as it has absorbed a certain quantity of moisture according to measurement or experience.

To that extent, instead of the hot air or accompanying the latter a vacuum being applied to achieve or intensify the splitting open of the cells and/or to achieve the reduction in the residual moisture, a vacuum with an absolute pressure of less than 0.25 bar and ideally in the region of 0.1 bar is preferably applied.

As a rule, apart from possible exceptions, no water, oil or other substance foreign to stone fruit or nut fruit seeds is added during the pre-crushing and as a rule also during the further grinding. This precisely reflects the strength of the method according to the invention. As a result of the exposure to hot air or vacuum and the release of the fats and oils stored in the cells accompanying the latter, the pre-crushing material already becomes pumpable. It is thus possible to pump the pre-crushing material through an agitator bead mill without further additives and in particular without the addition of water and/or oil. Very effective and precisely adjusted further crushing of the ingredients which form the batch composition is thus possible, often accompanied by a further increase in the pumpability of the batch composition.

It is especially preferred if the application of hot air or vacuum takes place in a horizontal drum mixer, which keeps the stone fruit or nut fruit seeds that are undergoing pre-crushing in permanent motion. In this way, the composition comprising the pre-crushed stone fruit or nut fruit seeds then also comes particularly intensively in contact with the hot air or the vacuum if it already has a tendency to form clumps under the influence of the withdrawal of the fat, oil or water from the cells, which is particularly the case when the composition has already been broken down by the pre-crushing into much smaller particles than for example mere nut or almond slices.

In this connection, it is particularly preferable that the application of hot air or vacuum is carried out in a conching device with a conching container at least essentially closed and a usually multi-arm conching tool preferably rotating therein on a horizontal shaft. Conching devices are known from chocolate production. They are also capable of venting such compositions and can be intensively integrated with hot air or vacuum, which are at first still sticky/doughy on account of their very high free fat content are therefore not yet pumpable.

The pre-crushing preferably takes place by a pre-crushing device in the form of a grinder, in particular a conical grinder. The latter comprises a crushing cone rotating with a—preferably vertically arranged—shaft, which rotates in a stationary crushing ring thereby forming a crushing gap. The surfaces forming the crushing gap between them typically have profiling forming a driver. This serves for the entry of the stone fruit or nut fruit seeds to be pre-crushed into the crushing gap and ensures that the stone fruit or nut fruit seeds “jam” in a form-fitting manner in the crushing gap and are then shattered into pieces as the crushing cone continues to rotate. Due to the fact that a crushing gap is worked with, and the crushing cone and the crushing ring nowhere come into contact, pre-crushing in any case does not take place to such an extent that the crushing gap can clog. In addition, the fineness of the particles that are the result of the pre-crushing can be well adjusted, whereby the distance is changed which keeps the grinding cones from one another.

Advantageously, the hot air or vacuum application takes place in a conching device with a conching container at least essentially closed and a usually multi-arm conching tool preferably rotating therein on a horizontal shaft.

After the composition obtained by the pre-crushing and hot air or vacuum application has become pumpable, the fine grinding takes place with the aid of an agitator bead mill—through which the composition can now be pumped. The fine grinding with an agitator mill leads to very uniform and fine grinding, which can be controlled well and carried out effectively.

Irrespective of the process claims as such, protection for a device for carrying out the process according to the disclosure is also claimed. The device is characterised in that it comprises a pre-crusher, a hot air or vacuum applicator and a fine grinding device.

The pre-crusher is equipped and adjusted such that it pre-crushes the stone fruit or nut fruit seeds to be treated, in such a way that a pumpable composition can be produced from it with the aid of the hot air and/or vacuum applicator, without the composition being heated into the region of the Maillard reaction. This composition can then be pumped through the agitator bead mill without further additives to increase the flowability.

### BRIEF DESCRIPTION OF THE DRAWINGS

FIG. 1 shows a plant according to the invention for smaller quantities, in which only one horizontal mixer is used in which the vacuum is applied.

FIG. 2 shows a cross-section through a grinder which can be used for this example of embodiment for the purpose of the pre-crushing.

FIG. 3 shows a plant according to the invention for larger quantities. It is characterised by two horizontal mixers operating in parallel, a vacuum being applied in both.

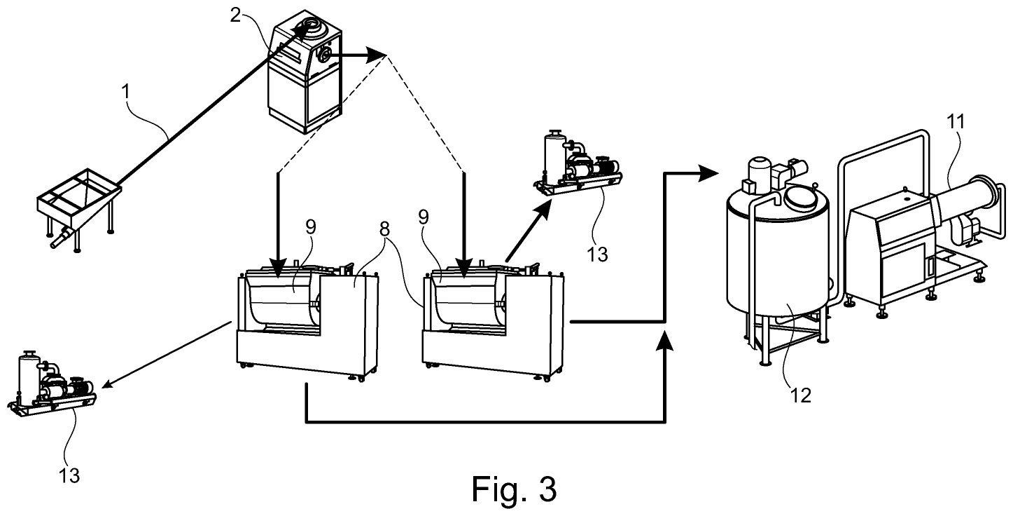

### DETAILED DESCRIPTION

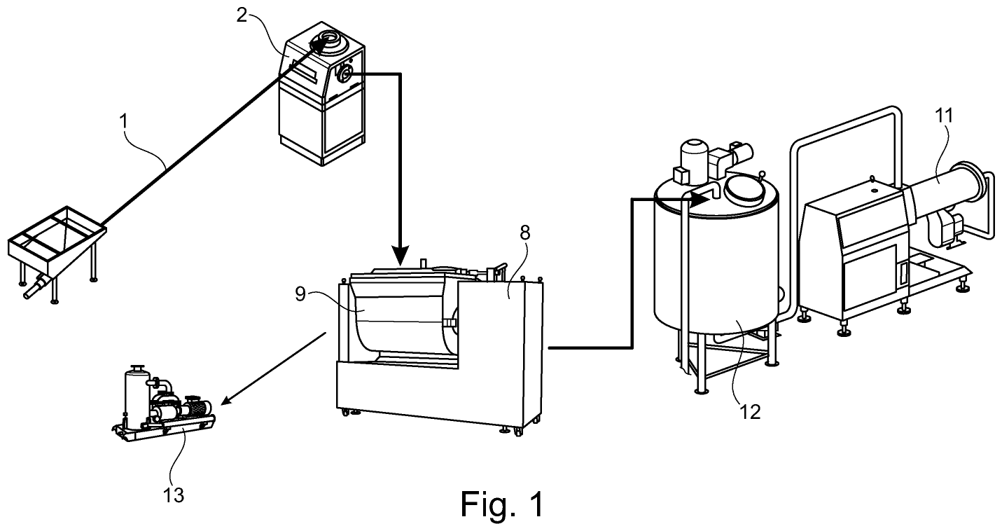

FIG. 1 shows a first example of embodiment of the invention, here using the example of hazel and/or cashew nuts and almonds. However, the description also applies accordingly to other nut seeds or stone fruit seeds.

The said nuts or almonds are delivered whole or sliced via a conveyor 1, usually a screw conveyor or a vibration conveyor, onto pre-crusher 2. A device can be used as a pre-crusher 2 such as is marketed for example by the company Netzsch under the name Mastercream®.

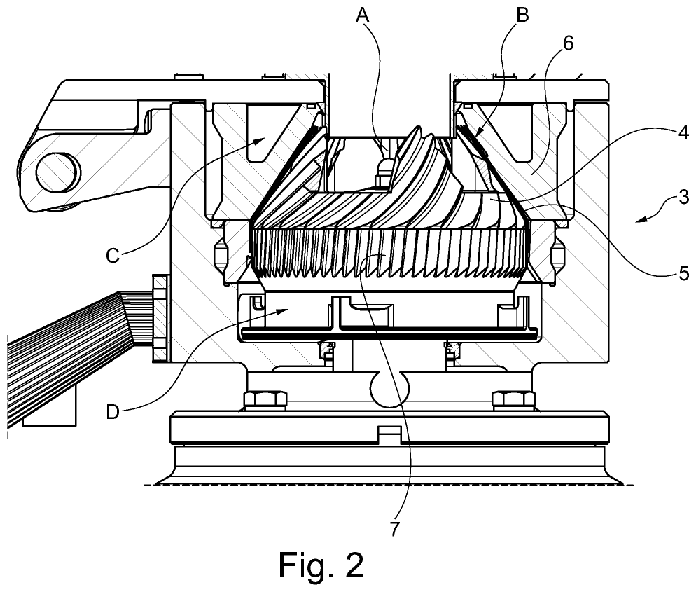

This pre-crusher is ideally constituted as a conical grinder 3, for example such as that shown in FIG. 2. Conical grinder 3 is fed in the vertical direction from above. The nuts and almonds to undergo pre-crushing fall on crushing core 4 tapering upwards. Crushing core 4 has a preferably helical profiling. This helical profiling catches hold of the incoming nuts and pulls them into crushing gap 5. This crushing gap is formed by crushing cone 4 together with stationary crushing ring 6, in which it rotates. The rotation takes place together with a vertical shaft. In this way, the nuts, or almonds to undergo pre-crushing are smashed to pieces in the crushing gap. From there they fall into the second part of the grinder. This second part is formed by a cylindrical grinding disc 7 adjoining the lower edge of the grinding core. Cylindrical grinding disc 7 also has a profiling which assists the grinding.

The grinding process is typically carried out in such a way that the temperature of the nuts or almonds to be pre-crushed remains below 110°, throughout the entire pre-crushing process. This prevents an influence on the flavour or even the formation of toasted aromas. In most cases, water cooling of the pre-crusher and its grinder can be dispensed with, instead of which a suitable adjustment of the gap width of the grinder and/or its shaft speed is reached, such that excessive heating does not takes place.

Pre-crusher 2 is preferably operated with a shaft speed of approximately 3000 to 4200 revs/min. Crushing cone 4 rotates with a correspondingly speed.

A viscous composition of nut or almond paste arises in the pre-crusher. The particles contained therein for the most part have an average particle diameter in the range from 30/100 mm to approximately 4/100 mm.

Although a part of the oils and fats stored in the cells has already been mobilised in this composition, the composition is not pumpable. If the clear diameter of the connecting line is not selected too small and the connecting line is implemented short, the finished pre-crushed composition can fall downwards into the horizontal mixer arranged below under the effect of gravity without further aids.

A conching device 8 is ideally used as a horizontal mixer, such as is known from chocolate production.

Such a conching device 8 can be designed similarly, for example, to the conching device described by the applicant in its German patent DE 10 2017 001 784 B4. It is thus made the subject-matter of the present description by way of reference.

A conching device is characterised by a conching container 9. A horizontal shaft rotates in the latter. A multi-arm conching tool 10 is usually attached to the shaft. Its arms plough up the doughy composition, which arises as a result of the pre-crushing, literally with each rotation. In this way, the composition is subjected very uniformly to the vacuum which is met in the conching container or the hot air, which is met there, which is applied in the conching device.

The conching container is typically connected to a vacuum pump. The vacuum pump partially evacuates the conching container. It thus places the latter under an under-pressure. This preferably takes place in such a way that less than 25% of the ambient pressure prevails in the conching container, ideally an under-pressure in the region of 0.1 bar, absolute, prevails.

The conching device works in the charge operation. It is fed by the pre-crusher until such time as it has reached its nominal loading. The pre-crushing process thus stops. The conching process device now begins to operate.

Precisely with the use of a vacuum, it has proved to be expedient if the conching device is preheated with hot water conveyed in separate channels and not therefore with the hot water coming into contact with the product to be conched. The preferred water temperature lies in the region of 60°+/−15° C., still better in a region of 90°+/−10° C. A conching container time of 60 to 120 minutes is typically selected. The conching tools preferably rotate with speeds of less than 20 revolutions/min.

After the passage of this time, a residual moisture of the conched material with a correct adjustment of the pre-crushing and conching parameters should be set in the range from 0.5%, +/−20%.

As a result of such conching or mixing under the influence of a vacuum, the cell walls of the composition removed from the pre-crusher, which until now are still intact, are broken open. In this way, fats and oils previously still stored in the cells and therefore held immobile are released. At the same time, the residual moisture is reduced. The latter, because the cell water is also mobilised. This then however transfers directly into the gas phase, unlike the mobilised oils and fats.

The viscosity of the composition thus diminishes markedly. The previously doughy-viscous composition becomes markedly more fluid. Its pumpability correspondingly increases. According to the inventor's findings, it is not the vacuum alone that is responsible for this effect. On the contrary, the combination of a vacuum and the permanent longer-lasting (usually more than 45 min) agitation of the composition by the mixer or the conching device appears to account for the decisive effect.

After the conching has completed, a composition is present which is already so flowable that it can be pumped into a storage container with the aid of a thick matter pump. Piston pumps are certainly possible here, but far from the ideal means of choice. The use of an eccentric screw pump has proved to be ideal, for example the Nemo series from the firm Netzsch. In any case, on account of its now improved pumpability, the composition that has arisen through the mixing or conching under vacuum and possible moderate thermal heat application can be pumped through an agitator bead mill 11.

Ideally, use is made of an agitator bead mill of the type Masterrefiner®, such as is marketed by the firm Netzsch. Grinding balls with a diameter in the range between 2 mm and 8 mm have proved to be most suitable—with this or other agitator bead mills. The dwell time of the composition in the agitator bead mill reasonably lies in the range from 2 to 4 hours, the agitator bead mill usually being passed through repeatedly.

In this way, a product, i.e. a batch composition, can be achieved in the agitator bead mill, the average particle size of which predominantly lies in the range between 1.5/100 mm and 10/100 mm. The last remains of previously still stored fats and oils are thus mobilised. A readily pumpable batch composition thus arises. Its viscosity which can be measured with the rotary viscosimeter preferably lies (and without the addition of foreign matter) in the range from 1.00E+2 cP to 1.00E+4 cP. Such a batch composition can readily be further processed with the standard metering pump in the food industry and precisely metered to the end product.

It should be stated at this point that aforementioned-storage container 12 is not absolutely necessary. It is however advantageous. This is because it enables agitator bead mill 11 to operate for a longer period than just for the short period in which the next charge is unloaded from conching device 8.

For larger through-put quantities, the previously described method is modified such that two or more horizontal mixers or conching devices 8 are operated in parallel. In this way, it can be ensured by using a correspondingly large storage container 12 that the device for fine grinding, i.e. the agitator bead mill 11 or the Masterrefiner, can be continuously loaded. This is because one of the two horizontal mixers or one of the two conching devices can always be loaded alternately, whilst the other works in parallel so that further product can be made available in a short time, which can be fed to the agitator bead mill.

A further example of embodiment is largely constructed precisely the same as the previously described examples of embodiment.

It differs from the latter, however, in that no vacuum is applied in the conching device. Instead, its conching container is supplied with hot air. The hot air has a sub-Maillard temperature ideally around 90° C. It comes directly into contact with the composition. It thus not only serves for the indirect heating of the conching container.

The dwell time in the conching container is in this case somewhat higher than in the vacuum application. It then amounts in many cases to approximately 90 to 180 minutes.

1. A method for preparing a pumpable batch composition of stone fruit and/or nut fruit seeds, in particular nuts, almonds or nut and almond mixtures, with the following steps:
Breaking open of the already pitted or stoned stone-fruit or nut-fruit seeds first takes place by pre-crushing;
A hot air and/or vacuum exposure of the composition arising from the pre-crushing then takes place for the purpose of breaking open its cells; and
at least a single-stage grinding of the composition arising from the pre-crushing and the hot air or vacuum exposure to form a pumpable batch composition.

2. The method for preparing a pumpable composition of stone fruit or nut fruit seeds according to claim 1, wherein the pre-crushing is pre-grinding, which preferably brings about a pre-grinding material, which essentially or at least predominantly has an average particle size in the range from 4/100 mm to 30/100 mm.

3. The method for preparing a pumpable composition of stone fruit or nut fruit seeds according to claim 1, wherein the hot air or vacuum exposure is in particular controlled via the exposure time, in such a way that the residual moisture is reduced to less than 2.1% and preferably to less than 1.85%, ideally to 0.5% +/−0.2%.

4. The method for preparing a pumpable composition of stone fruit or nut fruit seeds according to claim 1, wherein the air temperature of the hot air used for the drying lies in the region of over 60° and ideally in the region of over 75°.

5. The method for preparing a pumpable composition of stone fruit or nut fruit seeds according to claim 1, wherein the air temperature of the hot air used for the drying lies in the region of less than 100° and ideally in the region of less than 90°.

6. The method for preparing a pumpable composition of stone fruit or nut fruit seeds according to claim 1, wherein the hot air used for the drying flows through the drying area in such a way that it is conducted away from there still while the drying process continues to progress, as soon as it has absorbed a certain quantity of moisture according to measurement or experience.

7. The method for preparing a pumpable composition of stone fruit or nut fruit seeds according to claim 1, wherein for the drying a vacuum with an absolute pressure of less than 0.25 bar and preferably in the region of 0.1 bar is applied.

8. The method for preparing a pumpable composition of stone fruit or nut fruit seeds, in particular nuts and almonds according to claim 1, wherein no water, oil or other substance foreign to stone fruit or nut fruit seeds is added during the pre-grinding and as a rule also during the further grinding and as a rule also during the further grinding.

9. The method for preparing a pumpable composition of stone fruit or nut fruit seeds according to claim 1, wherein the application of hot air or vacuum takes place in a horizontal drum mixer, which keeps the stone fruit or nut fruit seeds undergoing pre-crushing in permanent motion.

10. The method for preparing a pumpable composition of stone fruit or nut fruit seeds according to claim 1, wherein the pre-crushing preferably takes place by a pre-crushing device, which includes a crushing cone rotating with a—preferably vertically arranged—shaft, which rotates in a stationary crushing ring thereby forming a crushing gap, wherein the surfaces forming the crushing gap between them are typically provided with a driver-forming profiling for the entry of the stone fruit or nut fruit seeds to be pre-ground into the crushing gap.

11. The method for preparing a pumpable composition of stone fruit or nut fruit seeds according to claim 1, wherein the application of hot air or vacuum is carried out in a conching device with a conching container at least essentially closed and a usually multi-arm conching tool preferably rotating therein on a horizontal shaft.

12. The method for preparing a pumpable composition of stone fruit or nut fruit seeds, in particular nuts or almonds according to claim 1, wherein the fine grinding takes place with the aid of an agitator bead mill, which preferably produces a final grinding material, which in solid phase has essentially or at least predominantly an average particle size in the range from 15/1000 mm to 10/1000 mm.

13. A device for carrying out the method of preparing a pumpable batch composition of stone fruit and/or nut fruit seeds, in particular nuts, almonds or nut and almond mixtures, with the following steps:
Breaking open of the already pitted or stoned stone-fruit or nut-fruit seeds first takes place by pre-crushing;
A hot air and/or vacuum exposure of the composition arising from the pre-crushing then takes place for the purpose of breaking open its cells; and
Next at least a single-stage grinding of the composition arising from the pre-crushing and the hot air or vacuum exposure to form a pumpable batch composition; and wherein the device comprises a pre-crusher, a hot air or vacuum applicator and a fine grinding device.
